(19) United States
(12) Patent Application Publication
(10) Pub. No.: US 2015/0216472 A1
(43) Pub. Date: Aug. 6, 2015

(54) USE OF DEVICE FOR MEASURING CERVICAL AND UTERINE ACTIVITY FOR DIAGNOSIS OF LABOR AND RELATED CONDITIONS

(71) Applicants: THE JOHNS HOPKINS UNIVERSITY, Baltimore, MD (US); CervoCheck, LLC, Baltimore, MD (US)

(72) Inventors: Abimbola Aina-Mumuney, Baltimore, MD (US); Karin Hwang, Ontario (CA); Nate S. Sunwoo, Laurel, MD (US)

(21) Appl. No.: 14/612,730

(22) Filed: Feb. 3, 2015

Related U.S. Application Data
(60) Provisional application No. 61/935,567, filed on Feb. 4, 2014.

(51) Int. Cl. A61B 5/00; A61B 5/0488

(52) U.S. Cl. CPC A61B 5/435; A61B 5/04882; A61B 5/4356; A61B 5/0084; A61B 5/6875

(57) ABSTRACT

Methods for detecting uterine and/or cervical activity in a subject are disclosed which comprise positioning a “uterine/cervical activity monitor” or “UCAM” within the subject, wherein at least one structural component of the UCAM is structured to be in contact with a cervical surface and/or a vaginal surface of the subject and which receives an electrical activity of said cervical surface and/or which receives electrical activity of the uterus through said vaginal surface and processing the electrical activity of said cervical and/or vaginal surfaces using a data processor in communication with the UCAM to detect uterine and/or cervical activity of the subject. The methods and UCAM device disclosed herein can be used to detect pre-term labor, differentiate non-labor contractions from “true” labor, as well as other indications.

### REFERENCE TO RELATED APPLICATIONS

This application claims the benefit of U.S. Provisional Patent Application No. 61/935,567, filed on Feb. 4, 2014, which is hereby incorporated by reference for all purposes as if fully set forth herein.

### STATEMENT OF GOVERNMENTAL INTEREST

This invention was made with government support under grant no. HD072684, awarded by the National Institutes of Health. The government has certain rights in the invention.

### BACKGROUND OF THE INVENTION

Despite recent technological medical breakthroughs, the issue of diagnosing preterm labor has continued to plague the obstetric community. In order to fully understand the gravity of this need, it is important to recognize the regrettable outcomes and heavy costs related to preterm birth. Preterm births lead to 70% percent of neonatal morbidity and mortality, and cost the United States over $26.2 billion in 2005 alone. Currently there is no way of accurately detecting preterm labor, which often leads to preterm birth.

Tocodynamometry (TOCO) is the mainstay for preterm labor evaluation but most clinicians would agree it has limited utility before 26 weeks gestation. The obesity epidemic has further reduced our ability to accurately detect uterine contractions using TOCO at any gestational age (GA) due to the excess tissue interfering with contraction detection. These issues prevent timely diagnosis and treatment of preterm labor. A method and device that detects preterm labor early in its course in patients is currently lacking.

Term delivery occurs between 37-42 weeks of gestation, whereas preterm delivery occurs between 20-37 weeks of gestation. Preterm delivery does not allow the fetus enough time to develop within the womb, resulting in severe short and long-term health issues for the neonate.

The unfortunate consequences of preterm delivery have encouraged the obstetric community to increase monitoring on those pregnancies with predetermined risk factors for preterm labor. These predetermined characteristics include factors such as extremes in maternal age (under 17 or over 35) and a history of preterm birth. Of the over 4 million births in the US each year, around 680,000 of those are considered at risk for preterm birth. These patients are monitored closely and required to make clinical visits as often as once a week. The main risk factors include: extremes in maternal age (<17 or >35); low socioeconomic status; stressful life situations; low weight gain; infection; cervical abnormalities or trauma; and history of preterm labor and birth.

All of the current methods available for detection of cervical and uterine activity are ineffective, insufficient, or inaccurate. Transvaginal ultrasound, while able to detect cervical changes even at very early gestational ages, cannot usually detect the contractions that are often present before these changes are evident. Symptomatic monitoring is insufficient because patients do not present for evaluation until the time for meaningful intervention has passed. Other tests, such as infection screening, only monitor one potential mechanism for labor initiation. Fetal fibronectin testing, while modestly accurate at predicting preterm labor, has a much higher negative predictive value.

Thus, there still exists an unmet need for a device that can accurately measure both cervical and uterine activity and which is not affected by gestational age, weight of the subject or other health issues.

### SUMMARY OF THE INVENTION

In accordance with an embodiment, the present invention provides a method for detecting uterine and/or cervical activity in a subject comprising: positioning a medical device within the subject, wherein at least one structural component of the medical device is structured to be in contact with a cervical surface and/or a vaginal surface of the subject; receiving an electrical activity of said cervical surface using a first electrode attached to said structural component, wherein said first electrode is in electrical contact with said cervical surface; and/or receiving an electrical activity of the uterus through said vaginal surface using a second electrode attached to said structural component, wherein said second electrode is in electrical contact with said vaginal surface; and processing the electrical activity of said cervical and/or vaginal surfaces using a data processor in communication with the medical device to detect uterine and/or cervical activity of the subject.

In accordance with an embodiment, the present invention provides a method for identifying whether a subject is suffering from pre-term labor comprising: a) positioning a UCAM within the subject with a gestational age of less than 37 weeks, wherein at least one structural component of the UCAM is structured to be in contact with a cervical surface and a vaginal surface of the subject; b) receiving an electrical activity of said cervical surface using a first electrode attached to said structural component, wherein said first electrode is in electrical contact with said cervical surface; receiving an electrical activity of the uterus through said vaginal surface using a second electrode attached to said structural component, wherein said second electrode is in electrical contact with said vaginal surface; c) processing the electrical activity of the electrodes in contact with the cervical and vaginal surfaces using a data processor in communication with the medical device to detect contractions of the uterus of the subject; d) determining the correlation of the waveforms of the signals of at least the cervical and vaginal surfaces; and e) identifying the subject as having pre-term labor wherein when the waveforms of the signals of the cervical and vaginal activity have a good positive correlation, or identifying the subject as not having pre-term labor wherein when the waveforms of the signals of the cervical and vaginal activity have a poor positive correlation.

In accordance with another embodiment, the present invention provides a method for prediction of the onset of labor in a subject comprising: a) positioning a UCAM within the subject having a gestational age of at least 37 weeks, wherein at least one structural component of the UCAM is structured to be in contact with a cervical surface and a vaginal surface of the subject; b) receiving an electrical activity of said cervical surface using a first electrode attached to said structural component, wherein said first electrode is in electrical contact with said cervical surface; c) receiving an electrical activity of the uterus through said vaginal surface using a second electrode attached to said structural component, wherein said second electrode is in electrical contact with said vaginal surface; d) processing the electrical activity of the electrodes in contact with the cervical and vaginal surfaces using a data processor in communication with the medical device to detect contractions of the uterus of the subject; e) determining the correlation of the waveforms of the signals of at least the cervical and vaginal surfaces; and f) identifying the subject as having true labor wherein when the waveforms of the signals of the cervical and vaginal activity have a good positive correlation, or identifying the subject as not having true labor wherein when the waveforms of the signals of the cervical and vaginal activity have a poor positive correlation.

In accordance with a further embodiment, the present invention provides a method for diagnosing a subject having chronic pelvic pain as being due to uterine contractions comprising: positioning a medical device within the subject, wherein at least one structural component of the medical device is structured to be in contact with a cervical surface and/or a vaginal surface of the subject; receiving an electrical activity of said cervical surface using a first electrode attached to said structural component, wherein said first electrode is in electrical contact with said cervical surface; and/or receiving an electrical activity of the uterus through said vaginal surface using a second electrode attached to said structural component, wherein said second electrode is in electrical contact with said vaginal surface; and processing the electrical activity of said cervical and vaginal surfaces using a data processor in communication with the medical device to detect contractions of the uterus of the subject wherein when the signals when the waveforms of the signals of the cervical and/or vaginal activity show contractions, the identification of the source of pelvic pain due to uterine contractions is made in the subject.

In accordance with an embodiment, the present invention provides a method for prognosing the likelihood of success of in-vitro fertilization (IVF) in a subject undergoing IVF treatment comprising: a) positioning a UCAM within the subject, wherein at least one structural component of the UCAM is structured to be in contact with a cervical surface and/or a vaginal surface of the subject; b) receiving an electrical activity of said cervical surface using a first electrode attached to said structural component, wherein said first electrode is in electrical contact with said cervical surface; and/or c) receiving an electrical activity of the uterus through said vaginal surface using a second electrode attached to said structural component, wherein said second electrode is in electrical contact with said vaginal surface; d) processing the electrical activity of said cervical and vaginal surfaces using a data processor in communication with the medical device to detect contractions of the uterus of the subject; e) determining the correlation of the waveforms of the signals of at least the cervical and vaginal surfaces; and f) identifying the subject as having a high likelihood of IVF failure due to rejection of implanted embryos when the signals when the waveforms of the signals of the cervical and/or vaginal activity show contractions.

### BRIEF DESCRIPTION OF THE DRAWINGS

FIG. 1 is a schematic illustration of a top perspective view of a medical device, in accordance with at least some embodiments of the present invention.

FIGS. 2A-2D are various schematic illustrations of the medical device, in accordance with at least some embodiments of the present invention.

FIG. 3 is schematic illustration of the placement of the electrodes on the patient, in accordance with at least some embodiments of the present invention.

FIG. 4 is a schematic illustration of the placement of the medical device within the patient, in accordance with at least some embodiments of the present invention.

FIG. 5 is a schematic illustration of a bottom perspective view of the medical device, in accordance with at least some embodiments of the present invention.

FIG. 6 is a schematic illustration of the stretch sensor that can be adapted for use, in accordance with at least some embodiments of the present invention.

FIG. 7A is a schematic illustration of a system for monitoring uterine and/or cervical activity indicative of labor in a patient, in accordance with at least some embodiments of the present invention.

FIG. 7B is a block diagram of the system operation, in accordance with at least some embodiments of the present invention.

FIG. 7C is a schematic illustration of a top view of an amplifier box attachable to a patient, in accordance with at least some embodiments of the present invention.

FIG. 7D is a schematic illustration of a side view of an amplifier box attachable to a patient, in accordance with at least some embodiments of the present invention

FIG. 8 is a circuit diagram of the amplifier box, in accordance with at least some embodiments of the present invention.

FIG. 9 is a software diagram of the data processor, in accordance with at least some embodiments of the present invention.

FIG. 10 shows an example of signal data on an external display, in accordance with at least some embodiments of the present invention.

FIG. 11 shows a chart illustrating the measurement of electrical activity of an example muscle using electromyography, in accordance with at least some embodiments of the present invention.

FIGS. 12-15 show comparison charts of measured data from exemplary cervical and vaginal surfaces, in accordance with at least some embodiments of the present invention.

FIG. 16 shows a graph of the data from the UCAM device of the present invention, TOCO, and IUPC with good correlation of uterine contraction measurement.

FIG. 17 is a graph of uterine activity as recorded by IUPC, vaginal electrode of the UCAM and TOCO in animals of light (upper tracing) or heavy (lower tracing) maternal weight.

FIG. 18 is a graph of uterine activity as recorded by IUPC, cervical electrode of the UCAM and vaginal electrode of the UCAM in animals in which delivery was either remote (upper tracing) or imminent (lower tracing).

### DETAILED DESCRIPTION OF THE INVENTION

In accordance with one or more embodiments, the methods of the present invention are used in conjunction with a medical device that is adapted to monitor uterine and/or cervical activity indicative of labor in a patient. This device was developed by the present inventors and is described in detail in U.S. Pat. No. 8,874,183, and is incorporated by reference herein as if set forth in its entirety.

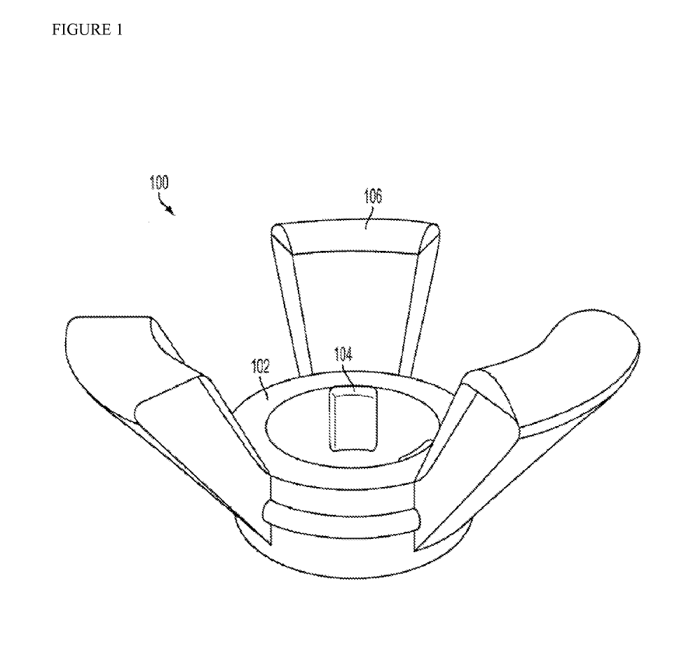

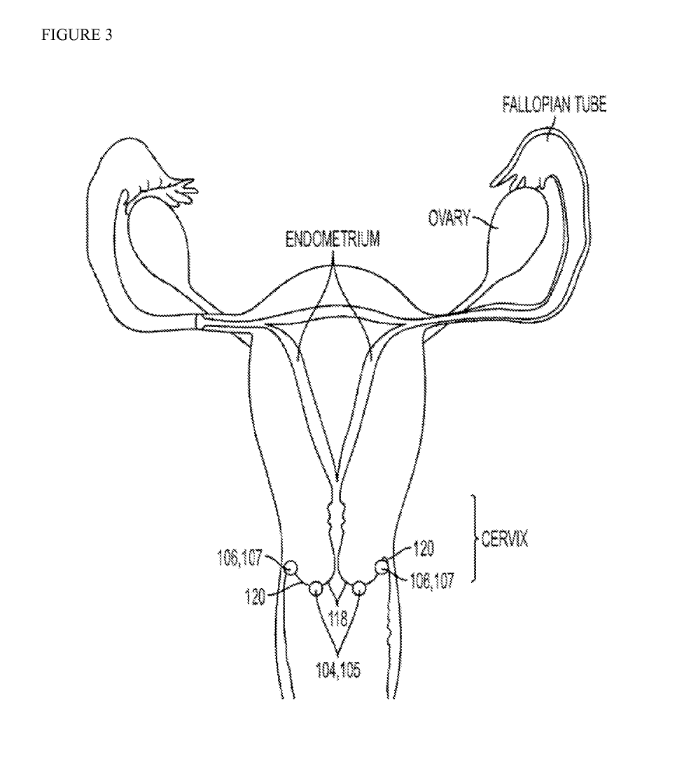

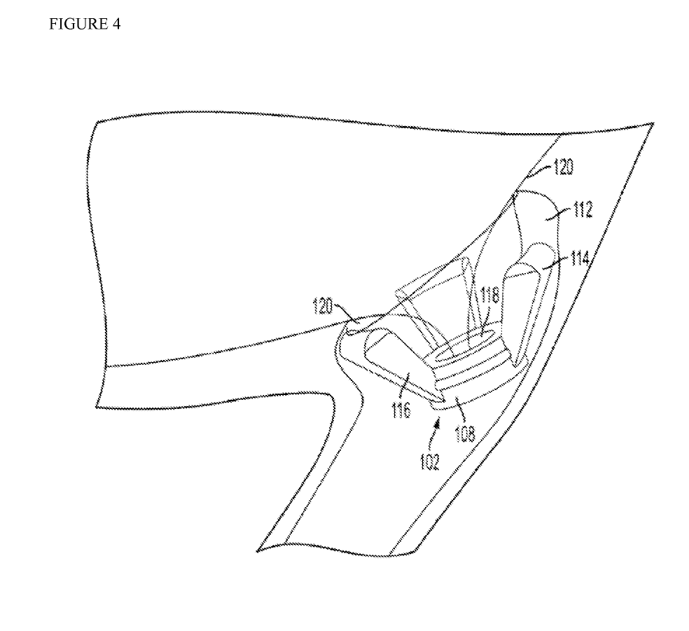

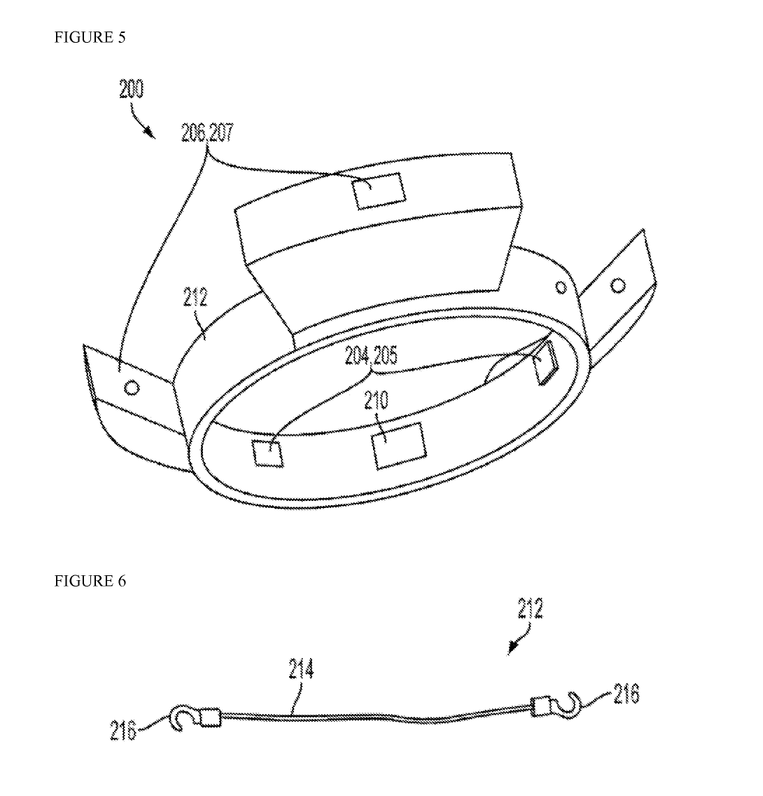

The device used in the inventive methods, is described briefly as follows. FIG. 1 is a schematic illustration of a top perspective view of a medical device 100, in accordance with at least some embodiments of the present invention. The medical device 100 is used in the inventive methods to monitor uterine and/or cervical activity (hereinafter, “uterine/cervical activity monitor” or “UCAM”) in a patient. The UCAM 100 includes a structural component 102, as well as a first electrode 104 and a second electrode 106 each attached to the structural component 102. The structural component 102 is structured to be in contact with a cervical surface and a vaginal surface of a medical patient (See FIGS. 3 and 4), such that the first electrode 104 is in electrical contact with the cervical surface and the second electrode 106 is in electrical contact with the vaginal surface. The first electrode 104 is adapted to receive an electrical activity of the cervical surface and the second electrode 106 is adapted to receive an electrical activity of the uterus through the vaginal surface.

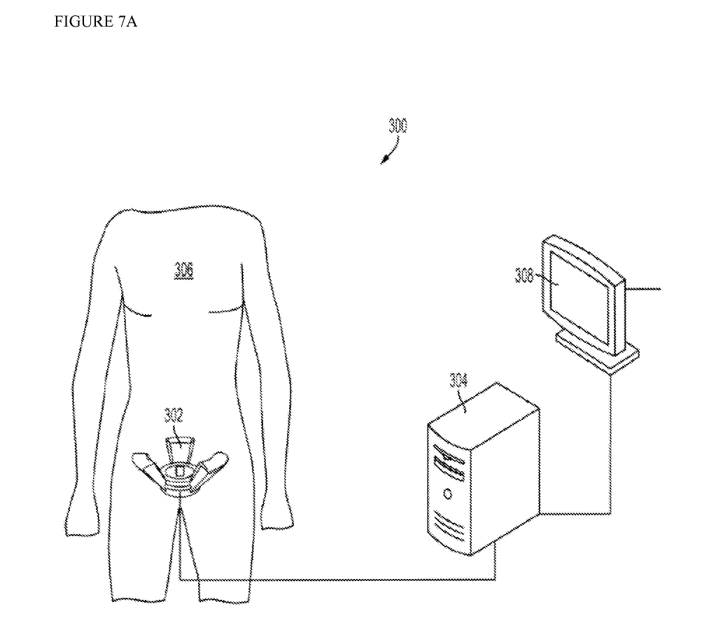

According to one embodiment of the UCAM used with the methods of the present invention, the first and second electrodes 104, 106 may comprise electromyography electrodes (hereinafter referred to as “EMG electrodes”). Electromyography (EMG) is a technique used for evaluating and recording electrical activity produced by muscles, for example the smooth muscle of the cervix, uterus and/or abdomen of a pregnant patient. (See FIG. 5 old, a chart illustrating the electrical activity of an example muscle using EMG). Alternatively, the device and methods can detect cervical and/or uterine activity using other types of biocompatible sensors.

In another embodiment of the UCAM used with the methods of the present invention, the first electrode 104 can receive the electrical activity of the cervical surface with respect to a reference signal and the second electrode 106 can receive the electrical activity of the uterus through the vaginal surface with respect to a reference signal. The reference signal may be, for example, the electrical activity taken from the inner thigh or other suitable location on the patient.

The electrodes are capable of remaining in contact with the vaginal fornix and the cervix and are capable of detecting electrical activity in the 0.001 to 1.0 Hertz range.

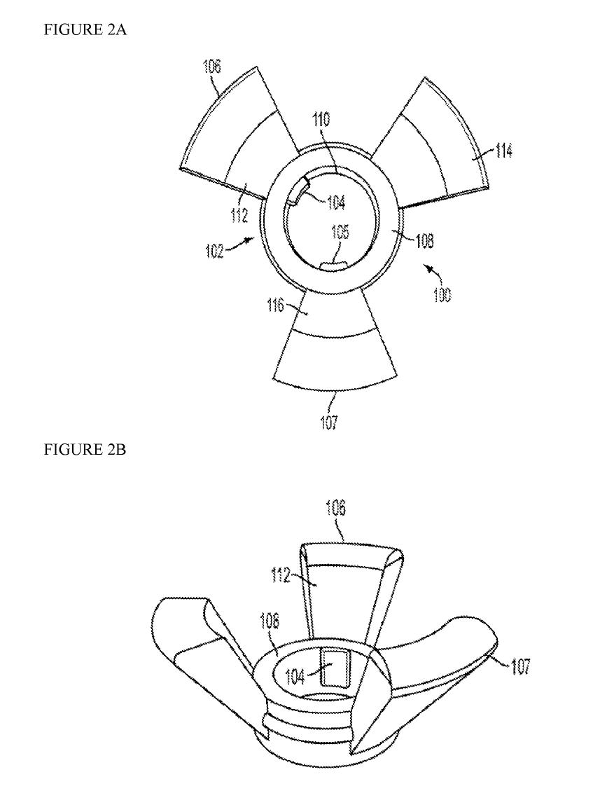

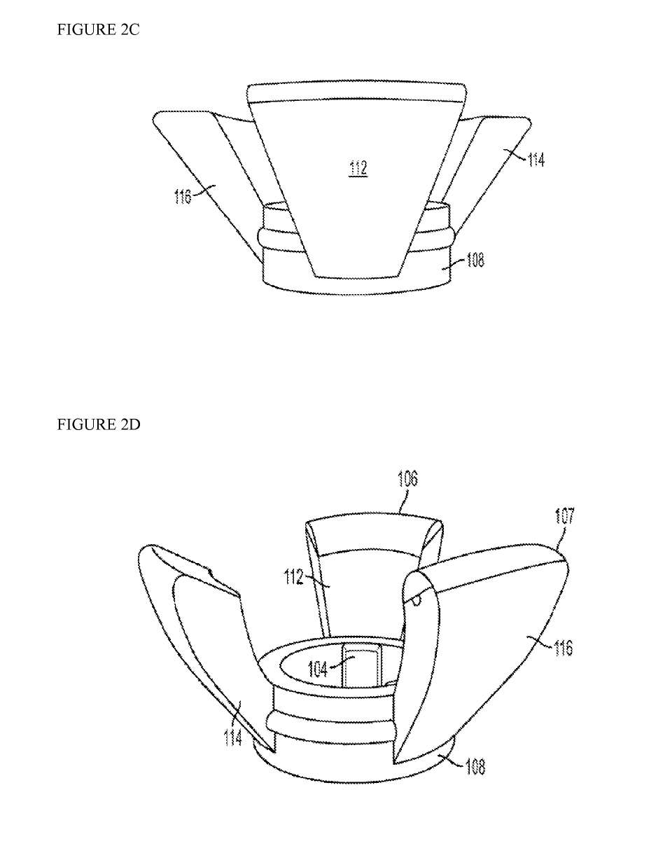

FIGS. 2A-2D are various schematic illustrations of the UCAM 100, in accordance with at least some embodiments of the present invention. As shown in the top view of FIG. 2A, the structural component 102 may include an elastic ring 108 defining a hollow center that is suitable to be arranged in contact with the cervical surface (See FIGS. 3 and 4). One or more electrodes 104, 105 may be positioned on an interior portion 110 of the elastic ring 108.

According to a further embodiment, the structural component 102 may also include a projecting portion 112 coupled to and/or integral with the elastic ring 108. The projecting portion 112 may be structured to be arranged in contact with the vaginal surface of the patient. The second electrode 106 may be positioned on the projecting portion 112.

According to another embodiment, the structural component 102 may include a plurality of projecting portions 112, 114, 116 coupled to and/or integral with the elastic ring 108. In this embodiment, electrodes 106, 107 may be coupled to each of the projecting portions.

FIG. 3 is schematic illustration to explain the desired placement of the electrodes of the UCAM on the patient, in accordance with at least some embodiments of the inventive methods.

FIG. 3 shows the patient's reproductive tract, including (from the top) the ovaries, the fallopian tubes, the endometrium (i.e. the inner membrane of the uterus), the cervix and the vagina of the patient. In this embodiment, two electrodes 104, 105 are placed in electrical contact with the cervical surface 118 of the patient and two electrodes 106, 107 are in electrical contact with the vaginal surface 120 of the patient.

FIG. 4 is a schematic illustration of the placement of the UCAM 100 within the patient, in accordance with at least some embodiments of the present invention. As shown, the structural component 102 is completely positioned within the reproductive tract of the patient. The elastic ring 108 of the UCAM 100 is positioned relative to a cervical surface 118 of the patient and the projecting portions 112, 114, 116 are positioned relative to a vaginal surface 120 of the patient.

FIG. 5 is a schematic illustration of a bottom perspective view of the UCAM 200, in accordance with at least some embodiments of the present invention. Similar to the embodiments described above, the UCAM 200 may include electrodes 204, 205 in electrical contact with the cervical surface 118 of the patient and electrodes 206, 207 in electrical contact with the vaginal surface 120 of the patient. Different numbers and placements of the electrodes are also possible.

According to one embodiment, a stretch sensor 212 (also referred to as a dilatation sensor or stretch gauge) may be attached to the UCAM 200. For example, the stretch sensor 212 may be attached along the exterior portion of the elastic ring 108 (See FIGS. 2A-2D). The stretch sensor 212 may be adapted to detect a change in resistance of the cervical surface 118 of the patient. In one embodiment, the stretch sensor may be a wire positioned along the exterior circumference of the elastic ring that carries a current. The stretch sensor 212 may measure physical changes from the stretching of two leads positioned at either end of the wire.

FIG. 6 is a schematic illustration of the stretch sensor 212 that can be adapted for use, in accordance with at least some embodiments of the present invention. According to one embodiment, the stretch sensor 212 may be a flexible component that changes resistance when stretched. When relaxed the sensor material may have a nominal resistance measured in ohms per linear inch. When stretched, the sensor's resistance may gradually increase. When the stretch sensor 212 is stretched to 50%, its resistance will approximately double. The stretch sensor 212 may measure stretch, displacement and force. According to one example, the stretch sensor 212 may be a flexible cylindrical cord 214 with spade or ring terminals 216 at each end. In the present application, the stretch sensor 212 may measure the dilatation of the cervical surface 118 of the patient.

According to another embodiment, as shown in FIG. 5, a light sensor 210 (also referred to as an effacement sensor) may be attached to the UCAM 200. For example, the light sensor 210 may be attached along the interior portion 110 of the elastic ring 108 (See FIGS. 2A-2D). The light sensor 210 may be adapted to measure changes in light reflectance and/or light transmission on the cervical surface 118 of the patient. The light sensor 210 may be one or more diodes for transmitting and/or receiving light. The term “light” is intended to have a broad meaning to include both visible and non-visible regions of the spectrum. For example, infrared, visible light and/or ultraviolet light emitting diodes (LEDs) can be used, depending on the particular embodiment. Optical diodes can be used to both transmit and receive in some embodiments, or there can be separate transmitters and receivers in other embodiments.

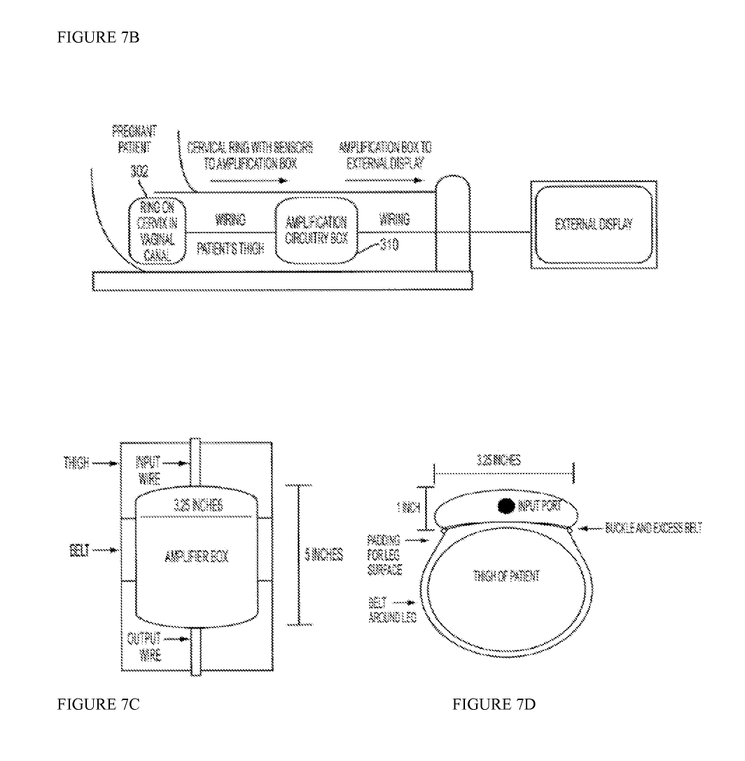

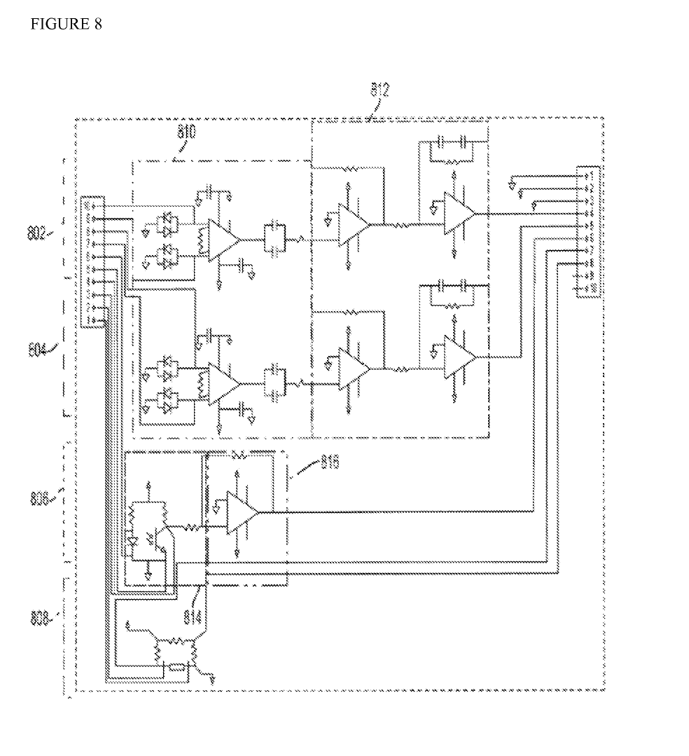

FIG. 7A is a schematic illustration of a system 300 utilizing the UCAM for monitoring uterine and/or cervical activity in a patient, in accordance with at least some embodiments of the present invention. The system includes software to perform amplification, filtering, and normalizing raw data and an external display for clinicians to examine processed data.

According to one embodiment of the inventive methods, the system 300 includes a UCAM 302 and a data processor 304 in communication with the UCAM 302. The UCAM 302 can be any one of the previous embodiments (See UCAM devices 100, 200 above) or a different embodiment. The data processor 304 is adapted to process the electrical activity of one or more electrodes in electrical connection with a cervical and/or vaginal surface to detect contractions on at least one surface of the patient 306. As described below, the data processor measures the voltage difference and/or electrical potential difference between electrodes. The electrodes may be in unipolar, bi-polar or multi-polar arrangement. The data processor offers real-time monitoring and signal processing. As discussed below, the system 300 can further include an external display 308 in communication with the data processor 304 to display information to a physician, patient or third party.

According to one embodiment of the inventive methods, the data processor 304 compares the electrical potential of the cervical surface 118 relative to the vaginal surface 120 to determine the uterine activity of the patient. In this embodiment, the medical device 100 requires a bi-polar arrangement, meaning only two electrodes. A first electrode 104 is in direct electrical contact with and receives the electrical activity of the cervical surface 118. A second electrode 106 is in direct electrical contact with and receives the electrical activity of the uterus through the vaginal surface 120 of a patient 306.

According to another embodiment of the inventive methods, the data processor 304 compares the electrical potential between at least two different locations of the cervical surface 118 to determine the uterine activity of the patient. In this embodiment, at least two electrodes 104, 105 are attached to the elastic ring of the medical device 302 to receive the electrical activity from at least two different locations of the cervical surface 118. The data processor 304 may then compare the electrical potential between the two different locations.

According to a further embodiment of the inventive methods, the data processor compares the electrical potential between at least two different locations of the vaginal surface 120 to determine the uterine activity of the patient. In this embodiment, at least one electrode 106, 107 is positioned on each of each projecting portion 112, 116 to receive electrical activity from at least two different locations of the vaginal surface 120. The data processor 304 then compares the electrical potential between the two different locations.

According to an alternative embodiment of the inventive methods, a stretch sensor 212 is attached to the UCAM 300 to detect changes in resistance in the cervical surface 118 of the patient. Alternatively, or additionally, a light sensor 210 may be attached to the UCAM 300 to measure light reflectance on the cervical surface of the patient. The data processor 304 is then be adapted to process the change in resistance and/or the reflectance of the cervical surface 118 to detect contractions of the uterus.

FIG. 7B is a block diagram of the system operation used in accordance with at least some embodiments of the methods of the present invention. According to this embodiment, an amplifier box 310 is in communication with the UCAM 302 via a hardwired or wireless connection. The amplifier box 310 is in further communication with a data processor 304 having an external display 308. As discussed below, the amplifier box 310 may include circuitry adapted to receive signals from each of the electrodes and sensors of the medical device 302, to amplify and reduce noise in the signals, and to output the signals to the data processor 304.

FIGS. 7C and 7D are schematic illustrations of an amplifier box attachable to a patient, in accordance with at least some embodiments of the present invention. In this embodiment, the amplifier box 310 may be fastened to the thigh of a patient using a sterilizable belt. The sterilizable belt may be secured by a sterilizable steel belt-buckle piece. Other fastening devices and securing means may be used. Similarly, the amplifier box 310 may be fastened at a different location on the patient or in close proximity to the patient, for example, on a medical bed or nightstand.

According to one embodiment, the amplifier box 310 may include an input wire in connection with the UCAM 302 and an output wire in connection with the data processor 304. Alternatively, data may be communicated into and out of the amplifier box 310 wirelessly.

According to another embodiment, the amplifier box 310 may be designed to fit comfortably around a patient's thigh or other body location. For example, the amplifier box 310 may have the approximate dimensions of 3 inches by 3.25 inches. Similarly, as shown in FIG. 7D, the sterilizable belt or other fastening device may include extra padding at the skin surface for additional comfort to the patient.

FIG. 8 is a circuit diagram of the amplifier box, in accordance with at least some embodiments of the present invention. The amplifier box 310 may be adapted to receive signals from each of the electrodes and sensors of the UCAM 302, to amplify and reduce noise in the signals, and to output the signals to the data processor 304. The amplifier box 310 may be housed within the data processing unit 304 or may be housed in a separate component in communication with the data processing unit 304, as shown in FIGS. 7C and 7D. The circuitry may be in communication with the UCAM 302 and the data processor 304 wirelessly or via hardwire. In an alternative embodiment, such filtering and signal processing may be done by software.

In FIG. 8, sections 802, 804, 808 and 810 show the communication connection between the electrodes and sensors of the UCAM 302 and the circuitry. For example, section 802 of the circuit diagram indicates the circuitry adapted to receive a signal from the cervical electrodes. Section 804 indicates the circuitry adapted to receive a signal from the vaginal electrodes. Sections 806 and 808 indicate the circuitry adapted to receive signals from the light sensors and the stretch sensors, respectively.

FIG. 8 identifies amplifier and band pass circuitry using blocks 810, 812, 814 and 816. Block 810 identifies amplification circuitry that can be further adapted by instrumentation amplifiers to amplify electrode signals with high common-mode rejection ratio (CMRR). Block 812 identifies band pass circuitry adapted to reduce noise in the electrode signals and smooth the output signal. Further, blocks 814 and 816 identify amplification circuitry adapted to amplify the signals received from light sensors. The circuitry may output the amplified and noise-reduced electrode and sensor signals to the data processor 304.

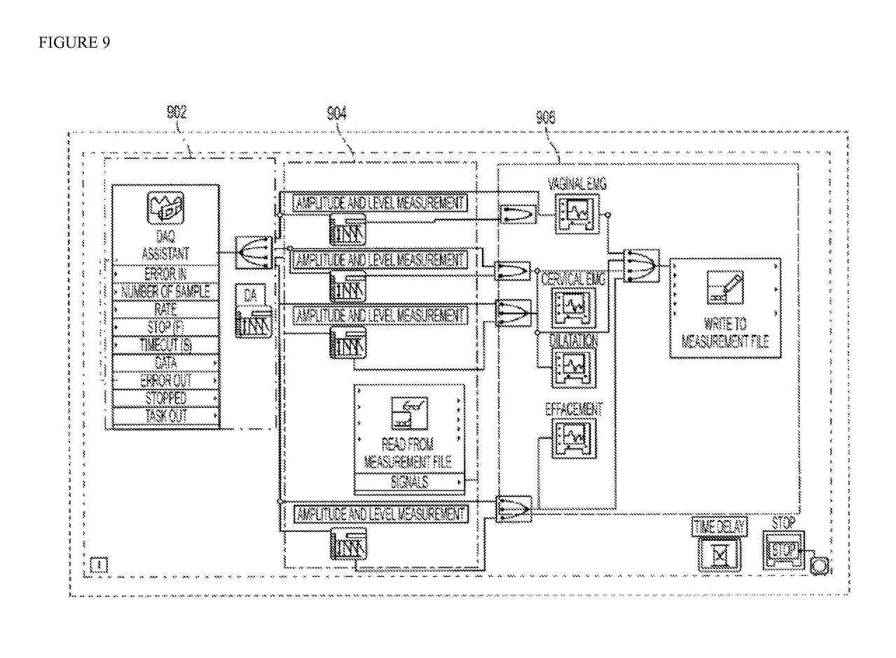

FIG. 9 is a software diagram of the data processor, in accordance with at least some embodiments of the present invention. Block 902 of the software diagram shows Data Acquisition Assistant software adapted to sample and filter input signals received from the UCAM 302. The Data Acquisition Assistant may perform various functions, including selecting the number of data values to sample. Block 904 shows software adapted to process signals to evaluate peak amplitude. Block 906 shows software adapted to save received data in serial text-based format. The software may further determine the magnitude of cervical changes for normalization based on previous exams and/or may link archived cervical information with current cervical measurements.

The data processor can be adapted to analyze the electrical activity of the first and second electrodes 104, 106 using vector hysterography (VHG). While vectors are an indispensable tool in physics and engineering, vectors have proven its usefulness in medicine as well with the advent of electrocardiography.

Living resting cells have an electrical double layer along their membranes, the positive charge along the external surface and the negative charge along the internal surface, creating what is known as an electric potential. Generally, a cell's resting state electric potential is negative. Depolarization (becoming more positive) and repolarization (returning back to resting state) of individual cells are the changes in the difference of these electrical charges across the cell membrane from the cell resting potential. Generally, these deviations are caused by the initiation of an action potential, presence of new molecules, or an electrical change in the environment. It is the polarization and depolarization of the cell membrane that moves electrical signals along tissues and organs, such as the uterus. Action potentials propagate rapidly throughout these organs and in the uterus, initiating movement of calcium into the cell via voltage-dependent channels, which activate myofilaments and generate the electromotive force. The force produced in a contraction is known to be caused by synchronization of multiple cells, the stimulation of their calcium gated ion channels, and the culmination of their myometrial activity.

In VHG, differences between the positive deflections and the negative deflections to the set of current measuring electrodes at the point of measurement may be plotted as a wave. The units on the axes are arbitrary, dependent on the position of the electrodes and the surface of contact. The length of the vector represents the mean electromotive force, while the angle between the vector and the zero-line represents the mean direction and the sense of the vector. This construction is based on vector addition. It is assumed that the mean electromotive force of the uterus is projected in the presence of at least two current measuring electrodes and the electrical axis may be constructed from this projection using vector addition. A derived vector may represent the projection of the true spatial vector upon a plane which is parallel to the surface of measurement.

The electrical axis at any given instant during a uterine contraction is continually changing in direction and magnitude and is called the instantaneous electrical axis. The instantaneous electrical axis of the whole uterus is a vector sum, the sum of the instantaneous electrical axes generated by the polarization and depolarization of the different parts of the uterus. The instantaneous electrical axis can be seen as an electric current, measurable by electrodes, indicative of the electromotive force. An electromotive force is a vector. Thus, what is detected by VHG may be considered waves of the uterus as depolarization and repolarization waves. Although linked to the initial action potential that initiated the chain of depolarization and repolarization of individual cells, VHG may provide a macroscopic view of the uterus by measuring the instantaneous electrical axis of the entire uterus, and not one cell or muscle fiber, relative to the plane of measurement.

According to one embodiment of the inventive methods, the UCAM 100 can apply at least one electric current measuring electrode to an abdominal, vaginal or cervical surface on a patient. The data processor processes and stores the electrical conductivity signal of the uterus, including the wave-front of electrical depolarization and repolarization, produced by the electrodes. Uterine activity is analyzed using parameters indicated from the wave.

The signals obtained by the cervical and/or uterine electrodes can be displayed in graphical form on any suitable media, including, a computer monitor, display, graph, for example.

There are two main phases of cervical activity: a latent phase and an active phase. A latent phase includes both synchronous bursts and asynchronous bursts. Synchronous bursts are a contraction response to an electrically-active uterus. Asynchronous bursts are generated by smooth muscles of an unripe cervix. In the active phase, the cervical electrical activity is reduced. Electrical activity of the cervix in the active phase is synchronous with uterine activity (i.e. the dominant force) and is indicative of effacement (restructuring) and dilation. Considering the behavior of the cervix in these two main phases, the value of additional vaginal and/or uterine electrodes lies not only in the strengthening of the data but also in its possible contributions to a more specific understanding of electrical activity as it travels from the uterus to the cervix during labor.

In an embodiment, the signals from the cervical and/or vaginal electrodes are superimposed onto the same graph with time along the x axis. Mean correlations between cervical and vaginal recordings with respect to number of contractions detected were compared and analyzed using the Student t test.

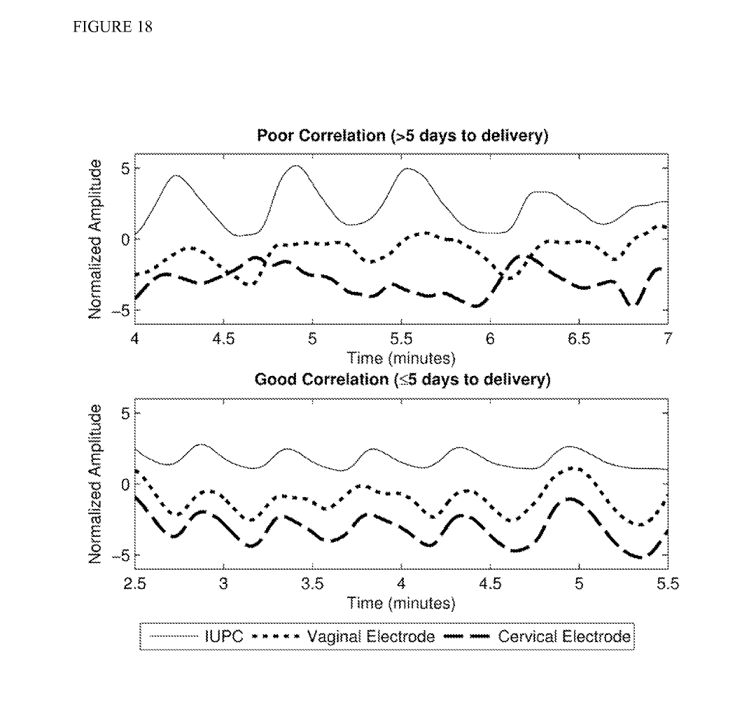

With respect to diagnosis of labor onset or pre-term labor, the signals from the UCAM can be used to differentiate asynchronous uterine contractions which are not indicative of “true” labor, from synchronous contractions indicative of “true” labor. As shown in FIG. 18, asynchronous contractions are identified as not indicative of labor when the waveform signals from the vaginal and cervical electrodes have lower statistical correlation. For example, without being held to any particular statistical cutoff value, having a correlation of 0.7 or less could indicate that the contractions are not “true” labor. For example, in some embodiments, the methods of the present invention can be used to identify false labor or not “true” labor, when the statistical correlation between the vaginal and cervical waveform signals are 0.6, 0.5, 0.4, 0.3, 0.2 or less.

Moreover, in accordance with an embodiment, the signals from the UCAM can be used to assess and identify “true” labor, when the waveform signals from the vaginal and cervical electrodes have a good statistical correlation, and/or increase in frequency over time. For example, without being held to any particular statistical cutoff value, having a correlation of greater than 0.7 could indicate “true” labor. For example, when the waveform signals from the vaginal and cervical electrodes have a statistical correlation of 0.7, 0.8, 0.9, 0.95 or greater can indicate “true” labor. Moreover, a combination of increasing positive statistical correlation of the waveform signals from the vaginal and cervical electrodes and increasing frequency of the waveform signals from the vaginal and cervical electrodes can indicate “true” labor.

According to one embodiment, the system and methods of the present invention may be used to monitor uterine activity indicative of early preterm labor with increased accuracy through direct application to the cervix. The system and method may measure and monitor the progression of uterine activity to identify cervical characteristics, dilatation and effacement, and therefore more accurately identify preterm labor. The system and method may also process the monitored data to assist in clinical diagnosis of preterm labor or pathologic and/or excessive uterine activity at any time during gestation. Preterm labor is defined as labor contractions prior to normal end of the gestational period. For example, prior to the 37th week of pregnancy in a human.

In accordance with an embodiment, the present invention provides a method for identifying whether a subject is suffering from pre-term labor comprising: a) positioning a UCAM within the subject with a gestational age of less than 37 weeks, wherein at least one structural component of the UCAM is structured to be in contact with a cervical surface and a vaginal surface of the subject; b) receiving an electrical activity of said cervical surface using a first electrode attached to said structural component, wherein said first electrode is in electrical contact with said cervical surface; receiving an electrical activity of the uterus through said vaginal surface using a second electrode attached to said structural component, wherein said second electrode is in electrical contact with said vaginal surface; c) processing the electrical activity of the electrodes in contact with the cervical and vaginal surfaces using a data processor in communication with the medical device to detect contractions of the uterus of the subject; d) determining the correlation of the waveforms of the signals of at least the cervical and vaginal surfaces; and e) identifying the subject as having pre-term labor wherein when the waveforms of the signals of the cervical and vaginal activity have a good positive correlation, or identifying the subject as not having pre-term labor wherein when the waveforms of the signals of the cervical and vaginal activity have a poor positive correlation.

According to another embodiment of the inventive methods, the UCAM 100 can apply a current measuring, multi-polar arrangement of electrodes to the surface of a patient, including the cervical or vaginal surface. The data processor processes and stores the time variation of a uterine electrical potential, detected by the electrodes. Alternatively, the data processor processes and stores the spatial variation of the uterine electrophysiological potential over time with the electrodes. The data processor then analyzes the uterine electrical potential and may display the uterine electrical potential in the form of a vector wave trace. The data processor may characterize the uterine and/or cervical contractility or electrical activity of the patient based on the analysis of the vector wave trace components.

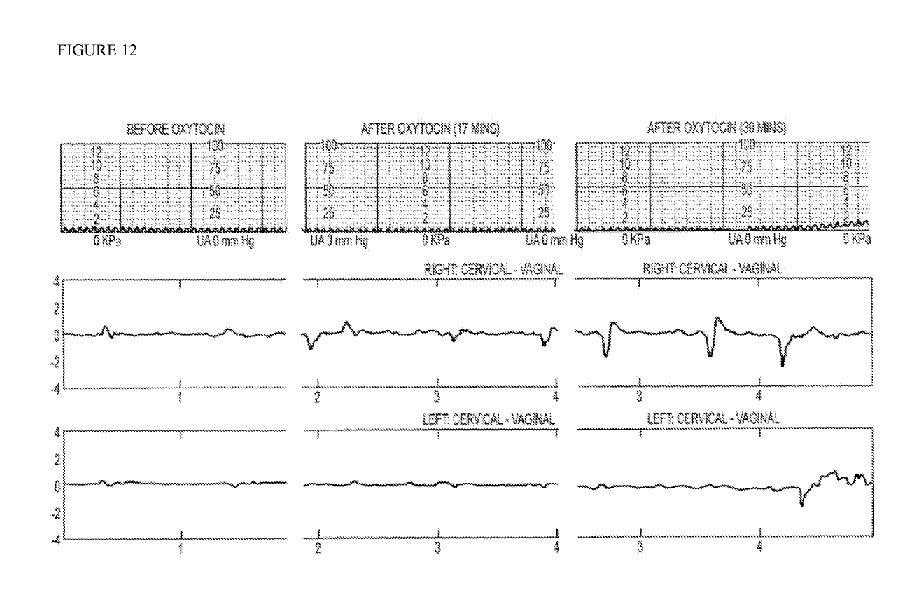

According to a further embodiment, the UCAM 100 can apply two or more electrical potential measuring electrodes to the surface of a patient, including the vaginal, abdominal or cervical surface. The data processor processes and stores the time variation of a uterine electrical potential, produced by the electrodes. The data processor can display the uterine electrical potential in the form of a wave trace (See FIGS. 12-15).

According to one embodiment of the inventive methods, the data processor using VHG may perform one or more of the following steps: 1) detecting electrical activity on the cervical and/or uterine surface of a subject and storing the activity as a signal; 2) calculating the amplitude of the potential vector in a stored signal; 3) comparing the calculated amplitude to a predetermine threshold; 4) calculating the frequency of the potential vector in the stored signals; 5) comparing the calculated frequency to a predetermined threshold; 6) calculating a rise time of a vector within said stored signal; 7) calculating the rate of rise of at least one of said vectors; 8) calculating a fall time of a vector within said stored signal; 9) calculating the rate of fall of at least one of said vectors: 10) examining one or more trends in uterine activity indicated parameters over time; and 11) displaying one or more trends in uterine activity indicated parameters over time.

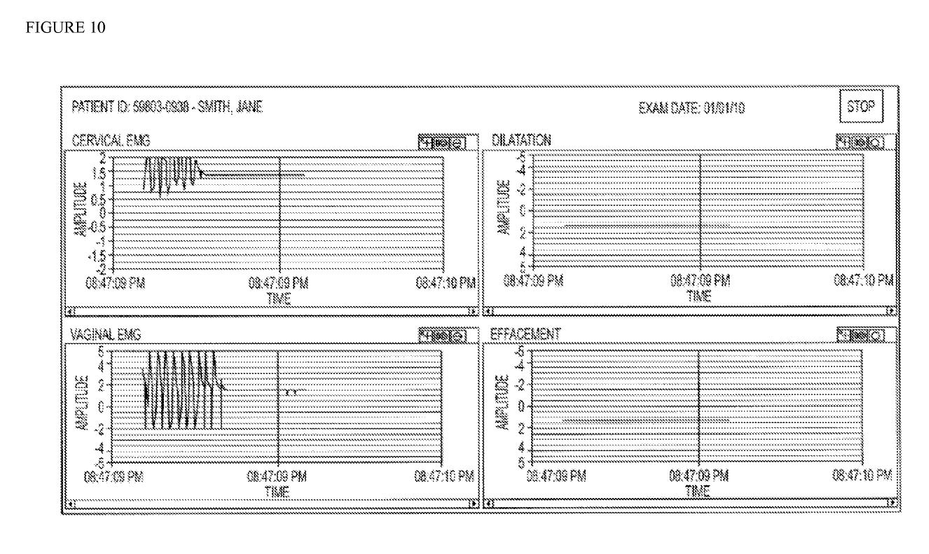

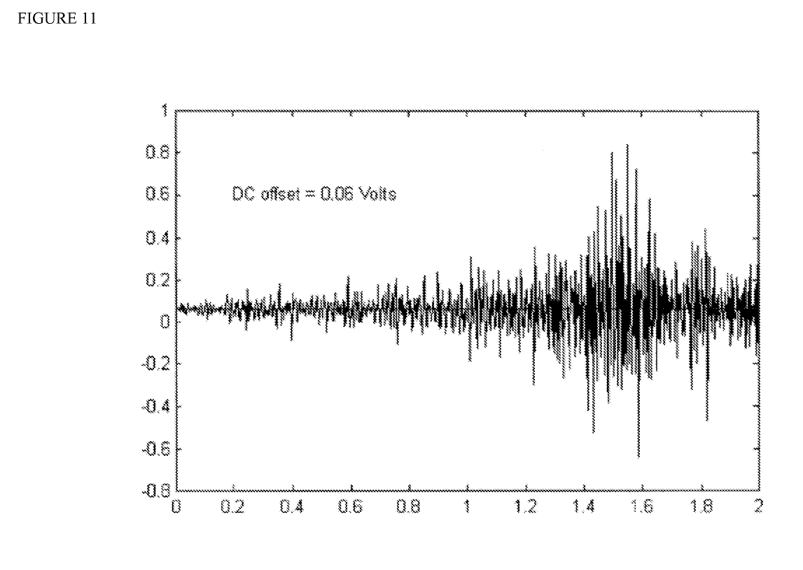

FIG. 10 is a schematic illustration of an external display in communication with the data processor, in accordance with at least some of the methods of the present invention. According to one embodiment, the data processor processes signal data into a smoothed and relevant signal for optimal diagnostic value and then transfer to and display through an external monitor. The outputs that address these inputs are realized through additional signal processing and smoothing in the software. The data are then transferred into a graphical display on an external monitor. As an example, the device processing and display may be done through a NI External Touch Screen monitor. Data may be communicated through any one of wireless, fiber optic, memory, hardwire, etc. The external display can convey information related to the data collected from each of the cervical electrodes, the vaginal electrodes, the light sensors and the stretch sensors.

According to one embodiment, a method of monitoring uterine and/or cervical activity indicative of labor in a patient includes the following steps: A UCAM 302 is positioned within a subject, where a structural component 102 of the UCAM 302 is arranged to be in contact with a cervical surface 118 and a vaginal surface 120 of the subject. A first electrode 104 attached to the structural component 102 receives an electrical activity of the cervical surface 118. A second electrode 106 attached to the structural component 102 receives an electrical activity of the uterus through the vaginal surface 120. A data processor 304 processes the electrical activity of the cervical and vaginal surfaces using a data processor to detect synchronous contractions of the uterus indicative of labor.

According to a different embodiment, the method for measuring contractions may include the steps of measuring cervical electrical potential directly from the cervical wall, measuring vaginal electrical potential from the vaginal wall, and extrapolating the data from the cervical and vaginal potentials to determine the changes in uterine contractility and the presence of uterine contractions.

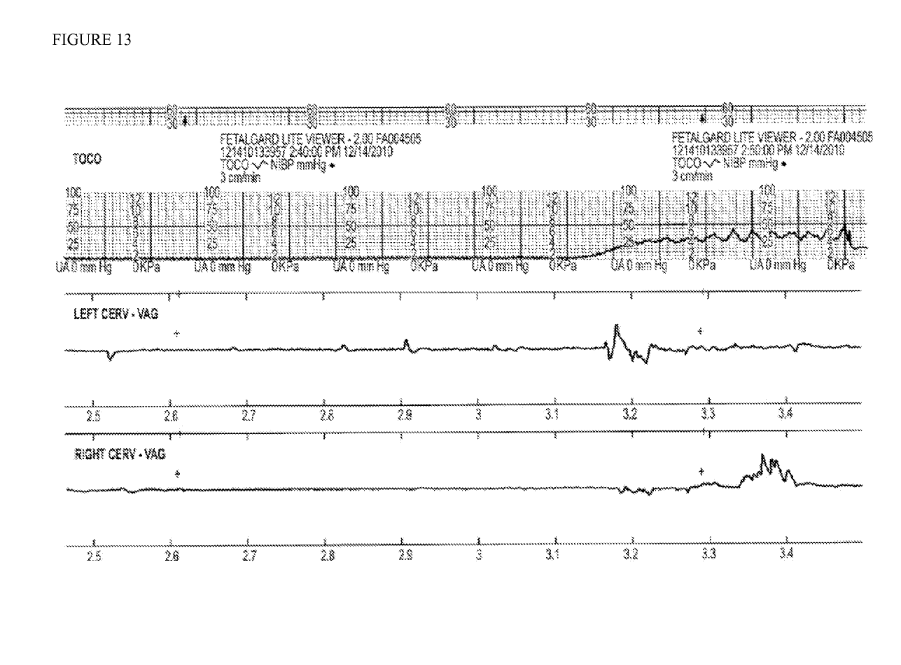

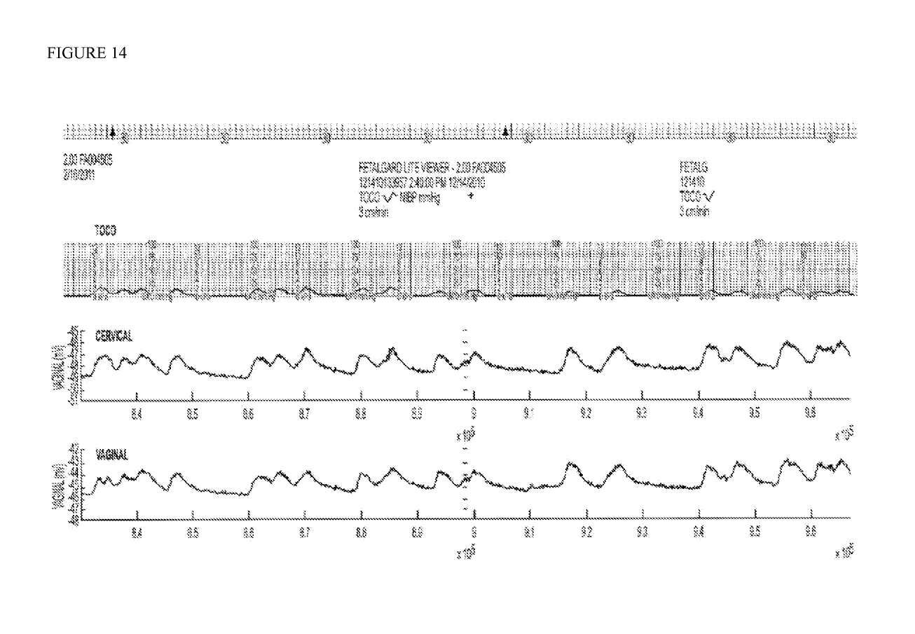

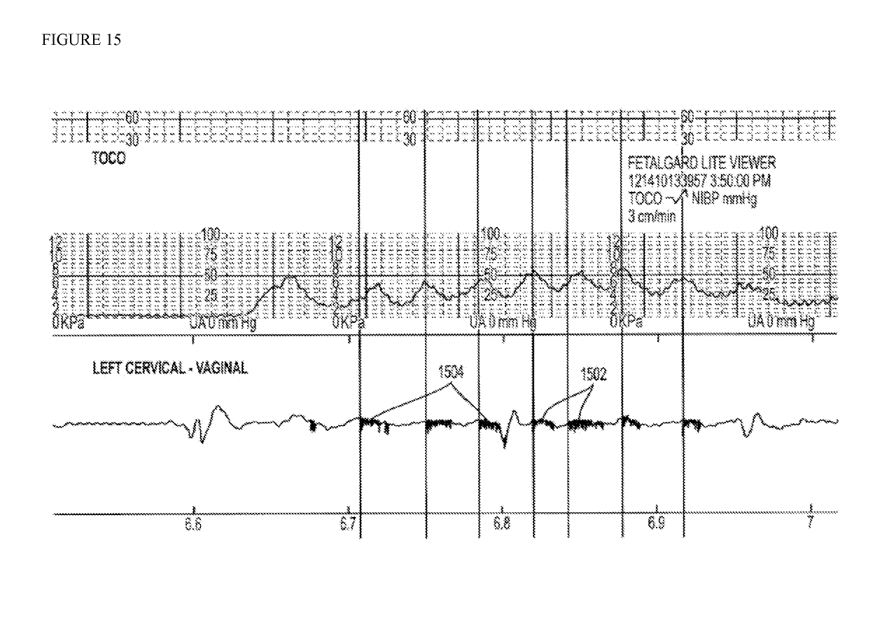

FIGS. 12-15 show comparison charts of measured data from exemplary cervical and vaginal surfaces, in accordance with at least some methods of the present invention. The embodiment of FIG. 12 shows a segment of data taken before injecting oxytocin, a hormone that is released in large amounts after distension of the cervix and uterus during labor, a segment of data taken 17 minutes after injecting oxytocin and a segment of data taken 39 minutes after the injection. The top graph depicts measurements taken using a tocodynamometer (TOCO), a method commonly used in the art, the middle graph depicts measurements taken between left cervical and vaginal electrodes using VHG analysis, and the bottom graph depicts measurements taken between right cervical and vaginal electrodes using VHG analysis. The middle and bottom graphs of FIG. 12, both using VHG analysis, show the clear progression and increase in signal amplitude from a period prior to oxytocin injection through a period 39 minutes after injection. Dissimilarly, the signal from the TOCO shows a much slower and less-noticeable reaction to the oxytocin injection in the patient. Similar results are shown in FIG. 13. The embodiment shown in FIG. 14 shows the alignment of measured activity between the TOCO and VHG.

The embodiment shown in FIG. 15 shows differences in measured data from exemplary cervical and vaginal surfaces using EMG and VHG methods. The high frequency bursts 1502 are EMG bursts and the small undulations 1504 are VHG signals. FIG. 9 shows that the undulations 1504 in VHG coincide with the signal from TOCO and EMG bursts 1502 happen at the peaks of the VHG waves. In VHG analysis, the electrical signals in tissues are sum of action potentials being triggered in each cell of a tissue. As the action potentials fire from each cell and travel down the uterus in a wave, the wave is what is picked up by the electrodes and called VHG. Right when the wave of action potentials is directly under an electrode, however, the electrode essentially becomes an EMG electrode and picks up the high frequency bursts 1502 of the action potentials.

In accordance with another embodiment, the present invention provides a method for prediction of the onset of labor in a subject comprising: a) positioning a UCAM within the subject having a gestational age of at least 37 weeks, wherein at least one structural component of the UCAM is structured to be in contact with a cervical surface and a vaginal surface of the subject; b) receiving an electrical activity of said cervical surface using a first electrode attached to said structural component, wherein said first electrode is in electrical contact with said cervical surface; c) receiving an electrical activity of the uterus through said vaginal surface using a second electrode attached to said structural component, wherein said second electrode is in electrical contact with said vaginal surface; d) processing the electrical activity of the electrodes in contact with the cervical and vaginal surfaces using a data processor in communication with the medical device to detect contractions of the uterus of the subject; e) determining the correlation of the waveforms of the signals of at least the cervical and vaginal surfaces; and f) identifying the subject as having true labor wherein when the waveforms of the signals of the cervical and vaginal activity have a good positive correlation, or identifying the subject as not having true labor wherein when the waveforms of the signals of the cervical and vaginal activity have a poor positive correlation.

It is known that uterine contractions during in vitro fertilization (IVF) are a cause of IVF failures in a subpopulation of women undergoing IVF, where other factors, such as egg and sperm quality, and other disease or uterine abnormalities are not an issue. See, for example, R. Fanchin, et al., Hum. Reproduction, 13:1968-1974 (1998), where they found a clear correlation between increased uterine contractions and decreased clinical pregnancy rates in groups of women undergoing IVF. The use of the UCAM with the inventive methods allows clinicians to measure uterine contractions prior to IVF implantation and determine a risk or probability of success or failure of the implantation procedure, and resulting clinical pregnancy. A determination of increased vaginal and/or cervical contractions in a subject undergoing IVF when compared to normative controls indicates that the subject undergoing IVF has a higher probability of potentially expelling the implanted embryos and having implantation and pregnancy failure than controls. Clinicians having such information can provide options for treatment of the subject, before, during and/or after IVF, with uterine relaxing compounds such as, for example, progesterone, β-mimetics, anti-prostaglandins, NO related compounds, barusiban and atosiban.

In accordance with an embodiment, the present invention provides a method for prognosing the likelihood of success of in-vitro fertilization (IVF) in a subject undergoing IVF treatment comprising: a) positioning a UCAM within the subject, wherein at least one structural component of the UCAM is structured to be in contact with a cervical surface and/or a vaginal surface of the subject; b) receiving an electrical activity of said cervical surface using a first electrode attached to said structural component, wherein said first electrode is in electrical contact with said cervical surface; and/or c) receiving an electrical activity of the uterus through said vaginal surface using a second electrode attached to said structural component, wherein said second electrode is in electrical contact with said vaginal surface; d) processing the electrical activity of said cervical and vaginal surfaces using a data processor in communication with the medical device to detect contractions of the uterus of the subject; e) determining the correlation of the waveforms of the signals of at least the cervical and vaginal surfaces; and f) identifying the subject as having a high likelihood of IVF failure due to rejection of implanted embryos when the signals when the waveforms of the signals of the cervical and/or vaginal activity show contractions.

In some embodiments of the method for prognosing the likelihood of success of in-vitro fertilization in a subject, the waveforms of the signals of the cervical and/or vaginal activities are compared to normative controls of subjects having successful IVF procedures.

In some embodiments of the inventive methods, the clinician can proscribe uterine relaxing drugs to increase the probability of successful implantation to the subject undergoing the procedure, either prior to, during, and/or post-IVF procedure.

As used herein, the term “subject” refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

The UCAM 100, 200, 302 used in the inventive methods can comprise uterine contractility sensors (measuring cervical and vaginal electrical potential), effacement sensors (measuring tissue thickness) and dilatation sensors (measuring diameter). The outputs that address these inputs are realized in the selection of the cervical ring sensors through the specified characteristics and placement of the selected electrodes, LED emitter and detector pair and embedded stretch gauge.

According to a further embodiment of the methods of the present invention, a vaginal electrode can obtain a signal through contact with the vaginal surface with respect to a reference signal. The cervical electrode may obtain a signal through contact with the cervical surface with respect to a reference signal. The reference signal may be taken at the inner thigh or other suitable surface of the patient.

### EXAMPLES

Nine healthy pregnant sheep sourced from three farms based on availability (Archer Farms Inc., Darlington, Md.; Robinson Services Inc., Mocksville, N.C.; Thomas D. Morris Inc., Reisterstown, Md.) were studied over the course of five months at the Johns Hopkins Research Animal Resources (Johns Hopkins Medical Institutes, Baltimore, Md.). Most sheep had two fetuses, but one was a singleton and another carried triplets. Sheep's weights ranged from 115 to 210 pounds as measured on the morning of each experimental session. Each sheep was tested more than once depending on their gestational age until they either gave birth or had to be euthanized due to adverse reactions to general anesthesia. At least one week was allowed to pass between each experimental session on an individual sheep in order to afford sufficient time to recover from anesthesia. Gestational ages of the sheep on the days of experiments ranged from 90 to 141 days. On average, sheep have a 145 day gestational period.

Each sheep was monitored for uterine activity via three different modalities simultaneously. The electrical activity (electrohysterography or EHG) was recorded by the UCAM1 system which consisted of a ring connected to a Biopac MP36R Data Acquisition system and its PC software, AcqKnowledge 4.1 (Biopac Systems Inc, Goleta, Calif.). The intrauterine pressure was assessed using Corometrics 120 Series Maternal/Fetal Monitor and its accompanying intrauterine pressure catheter (IUPC) (GE Healthcare, Little Chalfont, UK). The distortion of the abdomen caused by uterine contractions (tocodynamometry) was recorded using Analogic FETALGARD Lite fetal monitor (Analogic Corporation, Peabody, Mass.).

Sheep have a bicornuate uterus with one uterine horn on each side of the sheep abdomen as opposed to a single cavity centrally located in humans. For this reason, each sheep was scanned using ultrasonography to determine if one or both uterine horns contained a lamb. Most of the sheep we used in this study carried one fetus in each horn, and we elected to monitor the uterine horn on the right side of the sheep in these cases. In the event that the sheep carried a singleton or two fetuses within one uterine horn (for a total of three), the uterine horn with the single fetus was monitored.

EHG from the UCAM of the present invention was recorded from four locations using two AgCl electrodes placed externally on the sheep abdomen at the fundus and immediately lateral to the uterine horn of interest, and another two were placed internally, on the cervix and at the vaginal fornix attached to a silicone ring. Reference and ground electrodes were placed exteriorly on the pubic bone lateral to the introitus. All of the abdominal and external pelvic electrode sites were sheared and prepped with conductive gel prior to electrode placement.

After the electrodes were applied, the tocodynamometer (TOCO) was placed externally on the uterine horn of interest, and the IUPC was inserted into the vaginal canal and through the cervix and advanced into the space between the uterine wall and the amniotic sac (extracoelomic space).

Signal Acquisition and preprocessing. Each experimental session required the subject sheep to be under anesthesia for the entire duration. Baseline recordings from UCAM/TOCO/IUPC were obtained for at least 15 minutes at the beginning of each session. UCAM as recorded at the four sites (cervix, vaginal, fundal, and lateral abdominal) were acquired from AC-coupled electrodes at a sampling frequency of 1000 Hz, with an amplification factor of 1000, and an upper cut-off frequency of 200 Hz. Once the baseline recording was complete, oxytocin was infused intravenously to initiate contractions, adjusting the infusion rate gradually until the sheep was noted to have discrete contractions at 1-3 minute intervals. Oxytocin was infused via an intravenous line inserted into a vein on the sheep's ear and the recordings from UCAM/TOCO/IUPC continued uninterrupted for approximately 6 hours on average. Recordings were continued after discontinuation of the oxytocin infusion until either contractions abated or at least 30 minutes has elapsed.

Data recorded from the three devices were then low pass filtered to 0.1 Hz and high pass filtered to 0.002 Hz using fourth order Butterworth filters and then downsampled using the decimate function in MATLAB (MathWorks Inc., Natick, Mass.) to 4 samples per second. The data from the TOCO and the IUPC were also filtered using fourth order Butterworth low pass filters with cutoff frequency at 0.2 Hz.

Data Analysis. All recordings were superimposed onto the same graph with time along the x axis. The units of the y axis differed for each device as IUPC measures in mmHg, TOCO measures in cm of water and the UCAM device measures in millivolts (mV). The recordings of the IUPC were held as the “true” characteristic of the uterus at any given point in time. Due to the differing units of measurement, comparison of effectiveness between the TOCO or UCAM and IUPC was limited to the total number of contractions (defined as gradual deflections from the baseline) noted by each device in 10-minute intervals along the total tracing.

These 10-minute intervals were then isolated by device and presented to two independent reviewers who were blinded to the source device of each tracing. One reviewer was a former Labor and Delivery nurse with >25 years' experience and the other a board-certified perinatologist. The two reviewers were asked to count the number of contractions recorded on each tracing and record these numbers on a Microsoft Excel spreadsheet. The correlation between reviewers was calculated for each device by determining a Pearson coefficient. The number of contractions for each segment of each device used in final analysis was then calculated by averaging the number of contractions recorded by each reviewer for the segment in question.

Using the number of contractions recorded by IUPC as “true”, the Pearson correlation between the number of contractions recorded by TOCO and EHG was then calculated across all experiments.

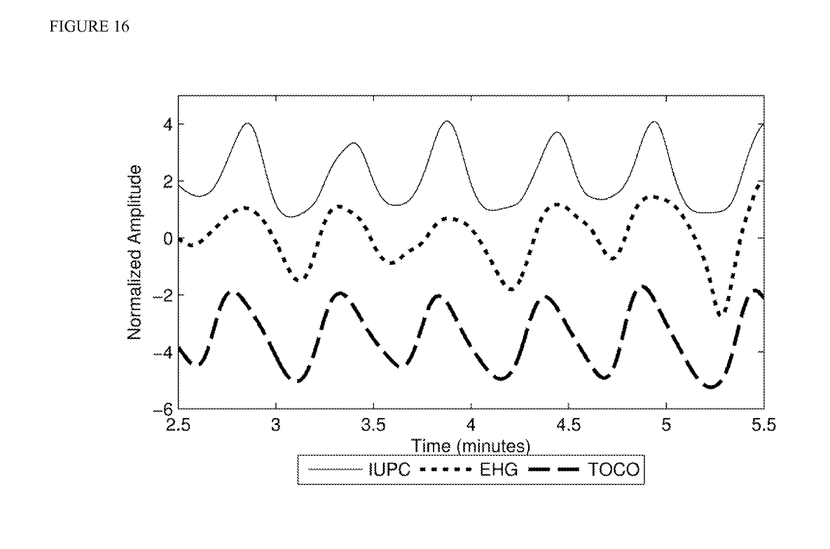

26 experiments were conducted in total. Data from 8 of these sessions were excluded from analysis due to an inability to record uterine activity with all 3 devices simultaneously. In all cases, this was due to an inability to advance the IUPC into the uterine cavity. Usually IUPC insertion was hindered by extremely tortuous cervical canals, but in one instance, insertion was precluded by torsion of the uterine horns due to a triplet gestation as revealed on necropsy. Good correlation was obtained and can be seen in FIG. 16.

### Example 1

Use of UCAM with a Patient. According to one embodiment, the UCAM 100, 200, 302 may be adapted to be applied directly to the cervix by the clinician. For example, when a symptomatic or high-risk patient visits her physician for a weekly or bi-weekly routine check-up, she may undergo a series of tests, including monitoring via a tocodynamometer, a digital examination and/or a transvaginal ultrasound to assess cervical length. This evaluation can last from two hours to 24 hours while the woman is probed and monitored. The disposable cervical elastic ring portion of the UCAM may be placed on the cervix of the patient after the initial digital examination, and remain for the full duration of the observation period. This may allow the physician to closely monitor the patient's dilatation, effacement, and contractions without constantly being in attendance. The readings may be provided by a separate monitor and saved to a hard-drive. At each evaluation for preterm labor, the patient may be provided with a new pre-sterilized cervical ring device. Aside from the placement of the UCAM, there will be no additional work for the physician other than to plug-and-monitor. After the evaluation, the disposable UCAM 100 can be discarded and the patient billed for the use of the UCAM.

The UCAM may be developed to fit directly into the current care pathway, allowing for quick adoption into existing obstetric practices. The initial target market for this device may include patients with known risk factors for preterm labor because they already undergo increased monitoring and would benefit most from an accurate monitoring device. By maintaining familiar display settings and simplified operation methods, the UCAM may easily be used by physicians in all pregnancies and may potentially replace current labor activity monitoring devices.

### Example 2

Direct Application to the Cervix. According to an embodiment, the UCAM 100, 200, 302 may be comprised of flexible, biocompatible and sterilizable components that conform to a normal pregnant cervix. The outputs that address these inputs may be realized in the design of the cervical ring through the specified characteristics of materials, fixation points and overall form factor.

According to one embodiment, the elastic ring 108 may have an initial inner diameter of approximately 20 mm and a stretched inner diameter of approximately 40 mm. The flexible band may have a thickness of approximately 4 mm and a depth of approximately 10 mm. Each wing portion may have a width of approximately 10 mm and a depth of approximately 7 mm.

According to another embodiment, the UCAM may be made of biocompatible medical grade polymer, for example, but not limited to, a medical grade silicone elastomer. The optimal Young's modulus, sometimes referred to as the elastic modulus or modulus of elasticity, may be approximately 1,500 to 15,000 psi. The tensile strength of the device may maintain fixation and contact with the interior surface region of the patient. Examples of such material are 1) MED-4025 silicone elastomer by NuSil², which has a tensile strength of 1272 psi, and 2) MED-4920 silicone elastomer by NuSil², which has a tensile strength of 1032 psi. Both materials have passed cytotoxicity testing and are adapted towards transfer/compression molding. It has been found, however, that silicone rubber is not approved for internal human use.

### Example 3

Direct Detection of Cervical/Uterine Activity. According to one embodiment, the UCAM 100, 200, 302 may be comprised of uterine contractility sensors (measuring cervical and vaginal electrical potential), effacement sensors (measuring tissue thickness) and dilatation sensors (measuring diameter). The outputs that address these inputs are realized in the selection of the cervical ring sensors through the specified characteristics and placement of the selected electrodes, LED emitter and detector pair and embedded stretch gauge.

According to another embodiment, unipolar electrodes may be used to detect both cervical and uterine contractions and/or electric potentials. Such electrodes may be made from, for example, 316L stainless steel and/or sintered silver chloride (Ag—AgCl). The electrodes may have an approximately 8 mm diameter. According to one embodiment, the electrodes may comprise EMG electrodes having a measurement range of approximately 50 to 3000 Hertz. According to a different embodiment, the electrodes may comprise biocompatible electrodes having a measurement range of approximately 0.001 to 0.5 Hertz and may be used for VHG applications. Alternatively, piezoelectric, fetal fibronectin and spring force sensors may be used to detect cervical and uterine contractions.

According to a further embodiment, a vaginal electrode may obtain a signal through contact with the vaginal surface with respect to a reference signal. The cervical electrode may obtain a signal through contact with the cervical surface with respect to a reference signal. The reference signal may be taken at the inner thigh of the patient.

According to another embodiment, light sensors may be used to determine the tissue thickness of the cervical or vaginal surface by obtaining a signal through light reflectance measurement from the surface. The light sensor may be less than 1.5 cm by 1.5 cm and may have a measurement range of 950 nm. According to an alternative embodiment, the light sensor may test the collagen of the blood in the surface tissue to determine efficacy (i.e. the shortening or thinning of the surface tissue). The light sensor may comprise one of a UV light emitter, an infrared light emitter or an LED light emitter. Similarly, the light sensor may utilize impedance, auto-florescence, ultrasound or reflection to detect tissue thickness of the cervical surface.

According to another embodiment, a stretch gauge may be used to determine the dilatation of the cervical surface. The stretch gauge may have, for example, a 7.5 mm radius and may measure approximately 1-2 K′Ω per linear inch. The stretch gauge may be placed directly around the circumference of the cervix. Alternatively, an ultrasound may be used to determine dilatation.

### Example 4

Special Considerations on Electrode Placement. According to one embodiment, the UCAM 100, 200, 302 may include only one set of electrodes placed on the inner surface of the cervical ring. However, the inclusion of additional electrodes may improve the resulting acquired signal. For example, the UCAM may include two additional electrodes attached at the tips of the wing protrusions of the cervical ring to pick up uterine contractions from the upper vaginal walls.

### Example 5

Real-time Monitoring and Signal Processing. According to one embodiment, multiple design inputs may be established requiring the device to optimally amplify and filter the acquired signals to provide the most useful and accurate information to the operating clinician. The outputs that address these inputs are realized in the development of the signal amplification circuitry, noise filtering band pass circuitry, and signal processing software.

For example, the system may utilize TI INA128P instrumentation amplifiers on all of the acquired signals. The acquired signals may then each be filtered through band pass circuitry built from LN741 CN op amps. These signals may then be processed using National Instruments LabView software in order to smooth the signal for post-processing display and save the obtained data for diagnostic reference and signal normalization. The current circuit and software diagrams are illustrated in FIGS. 8 and 9, respectively.

For amplification of the acquired signals, the circuit may utilize TI INA128P instrumentation amplifiers with a gain of 3000 and CMRR of 120. Although these amplifiers have been sufficient to pickup and display electrical signals from forearm muscle contractions, cervical and uterine electrical activity will be of much smaller magnitude and likely accompanied by various signal noise from the measurement environment. Thus, commercially available amplifiers, such as the CleveMed BioRadio, may also be used. Alternatively, the signal may be outsourced to a professional grade EMG amplifier built to the device's particular specifications.

In addition to amplification concerns, it is important to consider how the transmission of low-magnitude cervical and uterine electrical signals into the signal processing and filtering could be affected by signal-wire cross-talk. According to one embodiment, the circuit may utilize shielded wiring as well as twisted ground pair wiring schemes to limit cross-talk effects on signal propagation.

### Example 6

Additional Design Considerations and Constraints. The following represent other design considerations for the UCAM 100, including those of maintenance, compatibility, sterilization, regulatory requirements and labeling. For example, the UCAM may be adapted to withstand a three-foot fall and impact with concrete, wood or tile surface. The UCAM may be adapted not to interfere with digital exams intended to measure cervical changes. The UCAM 100 may also be adapted to be able to withstand in-package gamma ray sterilization before use without degrading or losing electrical signal function.

The UCAM may be adapted to follow all medical design controls, including software control and verification, as well as validation of all design inputs and outputs. The UCAM may also include instructions written to an 8^{th} grade reading level. The UCAM may further accommodate human factors, such as providing a cervical ring that is colored to provide a patient with options such as choice of pink, blue or a gender-neutral color like green.

In addition to these design goals, the following important constraints may be considered in the design of the system: 1) may not harm or damage mother and/or fetus; 2) may not cause any degree of cervical necrosis; 3) may not easily slip or fall away from cervix; 4) may not induce preterm labor; 5) may not contain Latex material; and 6) may not impede natural fluid flow.

### Example 7

Verification of Design Outputs. According to one embodiment, the UCAM 100, 200, 302 may be adapted towards the optimal materials and mixture ratio, the form for improved fixation and sensor placement, and flexible shielded wiring. For example, the form factor materials used in the UCAM may satisfy the input requirements of flexibility and sterilization, as has been determined through documentation and force measurements. The material may also be verified for biocompatibility. For example, the UCAM may comprise biocompatible and sterilizable materials made from a silicone elastomer.

Similarly, the form factor shape of the UCAM may be designed for optimal fixation and sensor placement in the target signal acquisition space. The basic design placement, as shown in FIGS. 3 and 4, has been initially verified using anatomy simulation models and confirmed by leading clinicians. For example, the UCAM may be adapted for optimal fixation in the target signal acquisition space using the following alternatives: memory foam, a cup with a hole, an inflatable balloon, a donut shaped balloon having a “U” shape, a claw with a spring, calipers and/or a spring sensor. The method of fixation may include spring force, a clip to the cervical wall, a screw hook, memory foam, an inflatable balloon (donut) and/or hydrogel.

According to a further embodiment, the sensors of the UCAM may be integrated into the device to specifically and efficiently receive the desired cervical and uterine activity. For example, the dilatation stretch gauge may be used in the device to pick up small changes in tension when applied to the gauge. In another example, the UCAM may include effacement sensors. Here, a UV light emitter/detector pair may be approved for biologic applications such as oximeter measurements. Alternatively, LED light emitters and/or infrared light emitters may be used. According to a further example, stainless-steel EMG sensors may be used in the UCAM to accurately pick up muscle contractions. The high-cost of these sensors has Jed to custom built steel electrodes, which, once fabricated, may be used to pick up contractions safely in vivo.

According to one embodiment, the signal amplification in the UCAM may use both commercially available amplifiers and outsourced fabricated amplifiers to accommodate the low order-of-magnitude electrical signals of the targeted cervical and vaginal surfaces. In addition to basic amplification verification, the UCAM 100 may limit potential crosstalk and use lubricating jelly, as described above. Further, software may be used at the data processor to reduce noise and common-mode rejection ratio (CMRR) of the signals, to optimize frequency filter and signal processing and to upgrade data acquisition (DAQ) sampling.

According to another embodiment, the data processor may transmit signal information to an external display. An example user interface may be provided using NI LabView software and the NI Industrial Touch Screen Monitor, as discussed above. The display may include human factors (such as the name of the patient) and a user interface to be used during in-clinic testing. Additionally, the fetal heart rate may be shown on the external display.

### Example 8

Alternative Uses of the UCAM. Additional uses of the UCAM include combination with fetal heart rate monitoring, chronic pelvic pain applications, monitoring of full term obese pregnant women, or monitoring of any pregnancy to obtain more accurate uterine contraction information. For example, the UCAM may be used to diagnose chronic pelvic pain in non-pregnant women, as some chronic pelvic pain is derived from uterine contractions, such as menstrual cramps. It can also be used in patients less than 20 weeks' gestation to determine if increased uterine activity is present and avoid unnecessary placement of a cerclage for an erroneous diagnosis of cervical incompetence.

### Example 9

Comparison of uterine activity of UCAM with activity recorded by IUPC and TOCO. The electrodes remain in contact with the vaginal fornix and the cervix to detect electrical activity in the 0.002-0.1 Hz range. Experiments were conducted using anesthetized pregnant Dorset ewes of varying gestational age and weights. A dilute oxytocin solution was infused and contractions were simultaneously recorded in 3 ways: 1) our silicone ring, 2) TOCO and 3) intrauterine pressure catheter (IUPC) placed in the extracoelomic space. The recorded activity from the ring and TOCO were compared to IUPC by calculating the Pearson correlation coefficient. Linear regression adjusted for effects of gestational age or weight over 18 experiments. Five of 6 sheep in this study were used for multiple experiments.

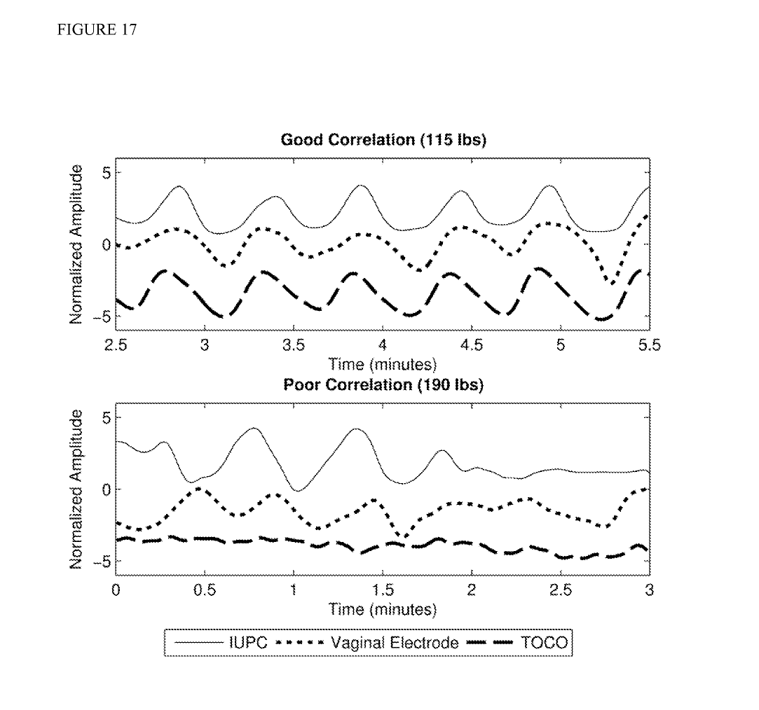

Sheep at 90-141 days gestation (term 145 days), weighing 115-210 lbs (average weight of mature Dorset ewe is 150-200 lbs) were used. Under ideal conditions, correlations between IUPC the UCAM and TOCO were as high as 0.94 and 0.91, respectively. Linear regression showed the correlations between IUPC and the UCAM were not affected by the sheep's gestational age or weight. The TOCO, in contrast, had an inverse relationship to the sheep's weight with increasingly poor correlation with IUPC as weight increased. Gestational age had no effect on TOCO (FIG. 17).

### Example 10

Examination of the correlation between recorded waveforms of vaginal and cervical electrical activity using the UCAM after inducing uterine contractions in an ovine model and comparing them with respect to the time to delivery.

The UCAM of the present invention was placed on the cervix of 6 sheep at varying gestational ages (GA) (90-141 days; term 145 days). One electrode was in contact with the cervix and one with the vaginal fornix to detect uterine contractions. Intravenous oxytocin was administered and an IUPC was placed in the extracoelomic space to detect uterine contractions (in mmHg) and serve as a control. Electrical activity between 0.002-0.1 Hz was recorded for ≧30 minutes. Data analyzed included GA of the sheep at time of recording and time to delivery. All 6 ewes delivered by 146 days. Recordings obtained ≦5 days to delivery were classified as “true labor”. Mean correlations between cervical and vaginal recordings with respect to number of contractions detected were compared and analyzed using the Student t test. Linear regression was used to adjust for effects of GA and individual sheep, since 5 of the 6 sheep were used for multiple experiments.

As seen in FIG. 18, the mean correlation between cervical and vaginal waveforms was significantly higher if the sheep was in “true labor” versus when it was far from delivery, 0.85±0.06 vs. 0.68±0.22, p=0.006. This difference persisted after adjusting for GA and the use of the same sheep over multiple experiments.

Cervical activity more closely resembles vaginal/uterine activity when delivery is imminent. The UCAM of the present invention detects this phenomenon non-invasively and can be useful clinically in identifying “true labor” during pregnancy, particularly in early gestation.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for identifying whether a subject is suffering from pre-term labor comprising:
a) positioning a UCAM within the subject with a gestational age of less than 37 weeks, wherein at least one structural component of the UCAM is structured to be in contact with a cervical surface and a vaginal surface of the subject;
b) receiving an electrical activity of said cervical surface using a first electrode attached to said structural component, wherein said first electrode is in electrical contact with said cervical surface; receiving an electrical activity of the uterus through said vaginal surface using a second electrode attached to said structural component, wherein said second electrode is in electrical contact with said vaginal surface;
c) processing the electrical activity of the electrodes in contact with the cervical and vaginal surfaces using a data processor in communication with the medical device to detect contractions of the uterus of the subject;
d) determining the correlation of the waveforms of the signals of at least the cervical and vaginal surfaces; and
e) identifying the subject as having pre-term labor wherein when the waveforms of the signals of the cervical and vaginal activity have a good positive correlation, or
identifying the subject as not having pre-term labor wherein when the waveforms of the signals of the cervical and vaginal activity have a poor positive correlation.

2. The method of claim 1, wherein the first and second electrodes comprise one of electromyography (EMG) electrodes or electrical potential measuring electrodes.

3. The method of claim 1, wherein the data processor is configured to process the electrical activity from the electrode to detect an electromotive force vector of a uterus of the patient from electrical activity in the range of approximately 0.001 to 0.5 Hertz on said surface of the patient, said electrical activity providing information that is indicative of labor.

4. The method of claim 1, wherein the data processor compares an electrical potential of the cervical surface relative to the vaginal surface to determine the uterine activity or changes in uterine contractility of the patient.

5. The method of claim 1, wherein the measuring component comprises an elastic ring defining a hollow center that is suitable to be arranged in contact with the cervical surface, wherein at least two electrodes are positioned on an interior portion of the elastic ring to receive an electrical activity of at least two different locations of the cervical surface.

6. The method of claim 1, wherein the data processor compares a difference in electrical potential between the at least two different locations of the cervical surface to determine the uterine activity of the patient.

7. The method of claim 1, wherein the UCAM further comprises a light sensor that is adapted to measure changes in reflectance of light from an interior surface of the patient.

8. The method of claim 7, wherein the light sensor comprises at least one light-emitting diode element.

9. The method of claim 1, wherein the UCAM further comprises a stretch sensor attached to the structural component that is adapted to detect a change in resistance of the cervical surface of the patient.

10. The method of claim 1, wherein the UCAM further comprises a reference electrode.

11. The method of claim 1, wherein the good positive correlation of the waveforms of the signals of at least the cervical and vaginal surfaces is at least 0.7 or greater.

12. The method of claim 1, wherein the poor positive correlation of the waveforms of the signals of at least the cervical and vaginal surfaces is at least 0.6 or less.

13. The method of claim 11, wherein the subject is informed of appropriate treatment options for pre-term labor.

14. A method for prediction of the onset of labor in a subject comprising:
a) positioning a UCAM within the subject having a gestational age of at least 37 weeks, wherein at least one structural component of the UCAM is structured to be in contact with a cervical surface and a vaginal surface of the subject;
b) receiving an electrical activity of said cervical surface using a first electrode attached to said structural component, wherein said first electrode is in electrical contact with said cervical surface;
c) receiving an electrical activity of the uterus through said vaginal surface using a second electrode attached to said structural component, wherein said second electrode is in electrical contact with said vaginal surface;
d) processing the electrical activity of the electrodes in contact with the cervical and vaginal surfaces using a data processor in communication with the medical device to detect contractions of the uterus of the subject;
e) determining the correlation of the waveforms of the signals of at least the cervical and vaginal surfaces; and
f) identifying the subject as having true labor wherein when the waveforms of the signals of the cervical and vaginal activity have a good positive correlation, or
identifying the subject as not having true labor wherein when the waveforms of the signals of the cervical and vaginal activity have a poor positive correlation.

15. The method of claim 14, wherein the first and second electrodes comprise one of electromyography (EMG) electrodes or electrical potential measuring electrodes.

16. The method of claim 14, wherein the data processor is configured to process the electrical activity from the electrode to detect an electromotive force vector of a uterus of the patient from electrical activity in the range of approximately 0.001 to 0.5 Hertz on said surface of the patient, said electrical activity providing information that is indicative of labor.

17. The method of claim 14, wherein the data processor compares an electrical potential of the cervical surface relative to the vaginal surface to determine the uterine activity or changes in uterine contractility of the patient.

18. The method of claim 14, wherein the measuring component comprises an elastic ring defining a hollow center that is suitable to be arranged in contact with the cervical surface, wherein at least two electrodes are positioned on an interior portion of the elastic ring to receive an electrical activity of at least two different locations of the cervical surface.

19. The method of claim 14, wherein the data processor compares a difference in electrical potential between the at least two different locations of the cervical surface to determine the uterine activity of the patient.

20. The method of claim 14, wherein the UCAM further comprises a light sensor that is adapted to measure changes in reflectance of light from an interior surface of the patient.

21. The method of claim 20, wherein the light sensor comprises at least one light-emitting diode element.

22. The method of claim 14, wherein the UCAM further comprises a stretch sensor attached to the structural component that is adapted to detect a change in resistance of the cervical surface of the patient.

23. The method of claim 14, wherein the UCAM further comprises a reference electrode.

24. The method of claim 14, wherein the good positive correlation of the waveforms of the signals of at least the cervical and vaginal surfaces is at least 0.7 or greater.

25. The method of claim 14, wherein the poor positive correlation of the waveforms of the signals of at least the cervical and vaginal surfaces is at least 0.6 or less.

26. The method of claim 24, wherein the subject is informed of appropriate delivery options for labor.

27. A method for prognosing the likelihood of success of in-vitro fertilization (IVF) in a subject undergoing IVF treatment comprising:
a) positioning a UCAM within the subject, wherein at least one structural component of the UCAM is structured to be in contact with a cervical surface and/or a vaginal surface of the subject;
b) receiving an electrical activity of said cervical surface using a first electrode attached to said structural component, wherein said first electrode is in electrical contact with said cervical surface; and/or
c) receiving an electrical activity of the uterus through said vaginal surface using a second electrode attached to said structural component, wherein said second electrode is in electrical contact with said vaginal surface;
d) processing the electrical activity of said cervical and vaginal surfaces using a data processor in communication with the medical device to detect contractions of the uterus of the subject;
e) determining the correlation of the waveforms of the signals of at least the cervical and vaginal surfaces; and
f) identifying the subject as having a high likelihood of IVF failure due to rejection of implanted embryos when the signals when the waveforms of the signals of the cervical and/or vaginal activity show contractions.

28. The method of claim 27, wherein the first and second electrodes comprise one of electromyography (EMG) electrodes or electrical potential measuring electrodes.

29. The method of claim 27, wherein the data processor is configured to process the electrical activity from the electrode to detect an electromotive force vector of a uterus of the patient from electrical activity in the range of approximately 0.001 to 0.5 Hertz on said surface of the patient, said electrical activity providing information that is indicative of labor.

30. The method of claim 27, wherein the data processor compares an electrical potential of the cervical surface relative to the vaginal surface to determine the uterine activity or changes in uterine contractility of the patient.

31. The method of claim 27, wherein the measuring component comprises an elastic ring defining a hollow center that is suitable to be arranged in contact with the cervical surface, wherein at least two electrodes are positioned on an interior portion of the elastic ring to receive an electrical activity of at least two different locations of the cervical surface.

32. The method of claim 27, wherein the data processor compares a difference in electrical potential between the at least two different locations of the cervical surface to determine the uterine activity of the patient.

33. The method of claim 27, wherein the UCAM further comprises a light sensor that is adapted to measure changes in reflectance of light from an interior surface of the patient.

34. The method of claim 33, wherein the light sensor comprises at least one light-emitting diode element.

35. The method of claim 27, wherein the UCAM further comprises a stretch sensor attached to the structural component that is adapted to detect a change in resistance of the cervical surface of the patient.

36. The method of claim 27, wherein the UCAM further comprises a reference electrode.

37. The method of claim 27, wherein the subject is informed of appropriate treatment options for inhibition of uterine contractions.
